(19) United States
(12) Patent Application Publication
(10) Pub. No.: US 2026/0008766 A1
(43) Pub. Date: Jan. 8, 2026

(54) EPISULFIDE COMPOUND AND OPTICAL MATERIAL COMPOSITION THEREOF

(71) Applicant: EFIRM NEW MATERIAL CO., LTD., Binzhou (CN)

(72) Inventors: Zongwang DING, Binzhou (CN); Wangen LIANG, Binzhou (CN); Weihua CUI, Binzhou (CN); Jianlin ZHANG, Binzhou (CN); Xianjun YI, Binzhou (CN); Yanli GAO, Binzhou (CN); Jinguo ZHANG, Binzhou (CN)

(73) Assignee: EFIRM NEW MATERIAL CO., LTD., Binzhou (CN)

(21) Appl. No.: 19/133,269

(22) PCT Filed: Mar. 28, 2024
(86) PCT No.: PCT/CN2024/084297

§ 371 Date: May 28, 2025

(30) Foreign Application Priority Data
Mar. 31, 2023 (CN) 202310364385.9

(51) Int. Cl. C07D 331/02; C08G 18/38; G02B 1/04

(52) U.S. Cl. CPC C07D 331/02; C08G 18/3874; G02B 1/041

(57) ABSTRACT

An episulfide compound and an optical material composition thereof are provided. The episulfide compound possesses a sulfoxide structure, and the presence of the polar group can effectively improve the alkali corrosion resistance of optical material substrates and reduce the rate of substrate burn, thereby improving substrate yield and reducing production costs.

### CROSS REFERENCE TO THE RELATED APPLICATIONS

This application is the national phase entry of International Application No. PCT/CN2024/084297, filed on Mar. 28, 2024, which is based upon and claims priority to Chinese Patent Application No. 202310364385.9, filed on Mar. 31, 2023, the entire contents of which are incorporated herein by reference.

### TECHNICAL FIELD

The present invention belongs to the field of new organic materials and optical materials, and relates to optical materials suitable for plastic lenses, prisms, optical fibers, information storage substrates, filters, etc., and more specifically to an episulfide compound and an optical material composition thereof.

### BACKGROUND

In recent years, with the development of optical resin technology, continuously improving the refractive index of optical resin lenses becomes the target pursued by future lenses. Sulfur-containing compounds, particularly poly-episulfide compounds and their formulation technologies, which can be used as raw materials for making resin lenses with an ultra-high refractive index, have been developed one after another. In the manufacturing process of optical resin lenses, after the primary curing is completed, release and mold opening are carried out, and the obtained substrate is edged and cleaned. The purpose of cleaning is to remove unreacted monomers and ground solid powder. In order to ensure the cleaning effect, a solution with a certain alkali concentration needs to be used, and the cleaning temperature shall be controlled at the same time. During the cleaning process, the resin lens will be corroded and burned due to the influence of alkali concentration or temperature, which will reduce the yield rate of the substrate and increase the production cost; and there is no technical solution recorded in the prior art on how to solve this problem.

Therefore, whether it is possible to provide an optical material with better performance and resistance to alkali corrosion has become one of the problems that need urgent solutions in this field.

### SUMMARY

In order to fill the gap in the prior art, the present invention provides an episulfide compound and an optical material composition thereof. The episulfide compound possesses a sulfoxide structure, and the presence of the polar group can effectively improve the alkali corrosion resistance of optical material substrates and reduce the rate of substrate burn, thereby improving substrate yield and reducing production costs; and the structure has the same difunctional episulfide structure as bis(β-epithiopropyl) sulfide, with little difference in the sulfur content, so it has no effect on other performance indicators such as the refractive index of the product.

The specific technical solution adopted in the present invention is as follows:

The inventors firstly provide an episulfide compound represented by formula (1):

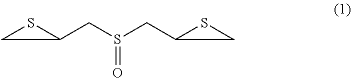

The method for preparing the episulfide compound represented by the above formula (1) may be exemplified by, but not limited to, the following method that: reacting the epoxy compound represented by formula (3) with a vulcanizing agent such as thiourea or thiocyanate under acidic conditions.

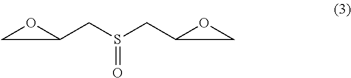

Preferably, the vulcanizing agent is selected from one or more of thiourea, potassium thiocyanate, ammonium thiocyanate, and sodium thiocyanate, preferably thiourea. The molar ratio of the vulcanizing agent dosage to the epoxy functional group in formula (3) is 1.0 to 3.0, preferably 2.0 to 2.5. When the molar ratio is less than 2.0, the raw material conversion rate is low, and when the molar ratio is greater than 2.5, the selectivity of the product shown in formula (1) is low, so neither is preferred.

The acid used in the reaction process can be listed as follows: inorganic acids such as nitric acid, hydrochloric acid, sulfuric acid, boric acid, arsenic acid, arsenous acid, pyroarsenic acid, phosphoric acid, phosphorous acid, hypophosphorous acid, hydrocyanic acid and chromic acid, and the like; organic carboxylic acids such as formic acid, acetic acid, peracetic acid, thioacetic acid, oxalic acid, tartaric acid, propionic acid, butyric acid, succinic acid, valeric acid, caproic acid, caprylic acid, naphthenic acid, methylmercaptopropionic acid, malonic acid, glutaric acid, adipic acid, cyclohexanecarboxylic acid, thiodipropionic acid, dithiodipropionic acid, maleic acid, benzoic acid, phenylacetic acid, o-toluic acid, m-toluic acid, p-toluic acid, salicylic acid, 2-methoxybenzoic acid, 3-methoxybenzoic acid, benzoylbenzoic acid, phthalic acid, isophthalic acid, terephthalic acid, salicylic acid, benzilic acid, naphthoic acid, acetic anhydride, propionic anhydride, butyric anhydride, succinic anhydride, maleic anhydride, benzoic anhydride, phthalic anhydride, pyromellitic anhydride, trimellitic anhydride and trifluoroacetic anhydride, and the like; phosphates such as mono-, di- and tri-methylphosphate; mono-, di- and tri-ethylphosphate; mono-, di- and tri-isobutyl phosphate; mono-, di- and tri-butylphosphate; and mono-, di- and tri-lauryl phosphate, and the like, including phosphites obtained as a result of the phosphate part of the above-listed substances being changed into phosphite; and also including dialkyldithiophosphate represented by dimethyldithiophosphate; phenols such as phenol, catechol, t-butylcatechol, 2,6-di-t-butylcresol, 2,6-di-t-butylethylphenol, resorcinol, hydroquinone, phloroglucin, pyrogallol, p-cresol, ethylphenol, butylphenol, nonylphenol, hydroxyphenylacetic acid, hydroxyphenylpropionic acid, amide hydroxyphenylacetate, methyl hydroxyphenylacetate, ethyl hydroxyphenylacetate, hydroxyphenetyl alcohol, hydroxyphenetyl amine, hydroxybenzaldehyde, phenylphenol, bisphenol A, 2,2′-methylene-bis(4-methyl-6-t-butylphenol), bisphenol F, bisphenol S, α-naphthol, β-naphthol, aminophenol, chlorophenol, 2,4,6-trichlorophenol, and the like; sulfonic acids such as methanesulfonic acid, ethanesulfonic acid, butanesulfonic acid, dodecanesulfonic acid, benzenesulfonic acid, o-toluenesulfonic acid, m-toluenesulfonic acid, p-toluenesulfonic acid, ethylbenzenesulfonic acid, butylbenzenesulfonic acid, dodecylbenzenesulfonic acid, p-phenolsulfonic acid, o-cresolsulfonic acid, aminobenzenesulfonic acid, sulphanilic acid, 4B-acid, diaminostilbenesulfonic acid, biphenylsulfonic acid, α-naphthalenesulfonic acid, β-naphthalenesulfonic acid, peri acid, Laurent's acid, phenyl J acid, and the like; etc. The acid may be used independently or as a mixture of two or more thereof.

Preferable examples of the acid are organic carboxylic acids such as formic acid, acetic acid, peracetic acid, thioacetic acid, oxalic acid, tartaric acid, propionic acid, butyric acid, succinic acid, valeric acid, caproic acid, caprylic acid, naphthenic acid, methylmercaptopropionic acid, malonic acid, glutaric acid, adipic acid, cyclohexanecarboxylic acid, thiodipropionic acid, dithiodipropionic acid, maleic acid, benzoic acid, phenylacetic acid, o-toluic acid, m-toluic acid, p-toluic acid, salicylic acid, 2-methoxybenzoic acid, 3-methoxybenzoic acid, benzoylbenzoic acid, phthalic acid, isophthalic acid, terephthalic acid, salicylic acid, benzilic acid, α-naphthoic acid, β-naphthoic acid, acetic anhydride, propionic anhydride, butyric anhydride, succinic anhydride, maleic anhydride, benzoic anhydride, phthalic anhydride, pyromellitic anhydride, trimellitic anhydride and trifluoroacetic anhydride, and the like.

More preferable examples of the acid are formic acid, acetic acid, peracetic acid, oxalic acid, tartaric acid, propionic acid, butyric acid, succinic acid, valeric acid, cetic anhydride, propionic anhydride, butyric anhydride, succinic anhydride, maleic anhydride, benzoic anhydride, phthalic anhydride, pyromellitic anhydride, trimellitic anhydride and trifluoroacetic anhydride; and the most preferred acid is acetic acid.

The molar ratio of the acid dosage to the epoxy functional group in formula (3) is 0.001 to 1.0, preferably 0.01 to 0.5. When the molar ratio is less than 0.01, the raw material conversion rate is low, and when the molar ratio is greater than 0.5, the selectivity of the episulfide compound shown in formula (1) is low, so neither is preferred.

In the above preparation method, the solvent is preferably used, and the reaction solvent is selected from alcohols such as methanol, ethanol, and the like; ethers such as diethylether, tetrahydrofuran, dioxane, and the like; hydroxyethers such as methylcellosolve, ethylcellosolve, butylcellosolve, and the like; aromatic hydrocarbons such as benzene, toluene, and the like; halogenation hydrocarbons such as dichloromethane, chloroform, chlorobenzene, and the like; water. Preferably, the solvent is selected from one of methanol, isopropanol, toluene, dichloromethane, or a mixture of two solvents thereof. There is no special requirement for volume of the solvent, as long as it can completely dissolve the sulfurizing agent.

The reaction temperature is usually selected to be 10° C. to 60° C., preferably 25° C. to 40° C. If it is lower than 10° C., the vulcanizing agent has poor solubility, while if it exceeds 60° C., polymers are generated, and side reactions increase.

After obtaining the above-mentioned episulfide compound, the inventors further provide an optical material composition. The composition includes a polymerizable compound mainly composed of a compound represented by formula (1) and a compound represented by formula (2), wherein the compound represented by formula (1) accounts for 0.001% to 6.0% by mass of the total amount of the optical material composition; more preferably 0.1% to 3% by mass; and the compound represented by formula (2):

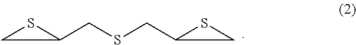

When the content of the compound in formula (1) is less than 0.001% by mass, the alkali resistance is poor. When the content exceeds 6.0% by mass, the impact resistance of the optical material is affected, and it is not conducive to processing and using in the later period. In addition, when the compound represented by formula (2) is used as the polymerizable compound, the compound represented by formula (2) in the optical material composition of the present invention accounts for 50% to 99.999% by mass of the total amount of the optical material composition, and more preferably 70% to 99% by mass.

The said polymerizable compound may include, in addition to the compound represented by formula (2), a thiol compound and an isocyanate compound.

When the total amount of the optical material composition is 100% by mass, the content of the thiol compound is usually 1% to 20% by mass, preferably 3% to 15% by mass, which can improve the heat resistance of the optical material; and when the content of the thiol compound exceeds 1% by mass, it can inhibit the yellowing of the lens during molding, and when the content is less than 20% by mass, it can prevent the reduction of heat resistance. The thiol compound used in the present invention can be used independently or as a mixture of two or more thereof.

Specific examples for the thiol compound include 2-mercaptoethanol, 3-mercaptopropanol, 1-mercapto-2-propanol, 1-hexanethiol, 1-octanethiol, bis(2-mercaptoethyl)sulfide, 2,5-dimercaptomethyl-1,4-dithiane, 1,3-bis(mercaptomethyl)benzene, 1,4-bis(mercaptomethyl)benzene, 4-mercaptomethyl-1,8-dimercapto-3,6-dithiaoctane, 4,8-dimercaptomethyl-1,11-dimercapto-3,6,9-trithiaundecane, 4,7-dimercaptomethyl-1,11-dimercapto-3,6,9-trithiaundecane, 5,7-dimercaptomethyl-1,11-dimercapto-3,6,9-trithiaundecane, 1,1,3,3-tetrakis(mercaptomethylthio)propane, pentaerythritoltetrakismercaptopropionate, pentaerythritoltetrakisthioglycolate, trimethylolpropanetristhioglycolate, and trimethylolpropanetrismercaptopropionate, preferably one or more of 2-mercaptoethanol, 3-mercaptopropanol, 1-mercapto-2-propanol, bis(2-mercaptoethyl)sulfide, and 4-mercaptomethyl-1,8-dimercapto-3,6-dithiaoctane.

When the total amount of the optical material composition is 100% by mass, the content of the isocyanate compound is usually 1% to 20% by mass, preferably 3% to 15% by mass. When the content of the isocyanate compound exceeds 1% by mass, the strength of the optical material is increased, and when the content is less than 20% by mass, the tone reduction can be inhibited. The isocyanate compound used in the present invention can be used independently or as a mixture of two or more thereof.

Preferably, the isocyanate compound contains at least two isocyanate groups, and the isocyanate compound is selected from diethylenediisocyanate, tetramethylenediisocyanate, hexamethylenediisocyanate, trimethylhexamethylenediisocyanate, cyclohexanediisocyanate, 1,3-bis(isocyanatemethyl)cyclohexane, 1,4-bis(isocyanatemethyl)cyclohexane, isophoronediisocyanate, 2,6-bis(isocyanatemethyl)decahydronaphthalene, tolylenediisocyanate, o-tolidinediisocyanate, diphenylmethanediisocyanate, diphenyletherdiisocyanate, 2,2′-bis(4-isocyanatephenyl)propane, triphenylmethanetriisocyanate, bis(diisocyanatetolyl)phenylmethane, 1,3-phenylenediisocyanate, 1,4-phenylenediisocyanate, 4,4′-diisocyanatebiphenyl, dicyclohexylmethane-4,4′-diisocyanate, 1,1′-methylenebis(4-isocyanatebenzene), m-xylylenediisocyanate, p-xylylenediisocyanate, m-tetramethylxylylenediisocyanate, p-tetramethylxylylenediisocyanate, bis(isocyanatemethyl)norbornene, bis(isocyanatemethyl)adamantane, thiodiethyl isocyanate, thiodipropyldiisocyanate, and thiodihexyldiisocyanate; preferably isophoronediisocyanate, m-xylylenediisocyanate, and 1,3-bis(isocyanatemethyl)cyclohexane.

Adding the above-mentioned thiol compounds and isocyanate compounds to the polymerizable compound mainly containing the compounds represented by formula (1) and formula (2) will not affect the alkali corrosion resistance of the final material.

Based on the above optical material composition, the inventors also provide a polymerizable and curable composition, which includes any one of the above optical material compositions and 0.01% to 1% by mass of a polymerization catalyst relative to the total amount 100% by mass of the optical material composition. The polymerization catalyst may be an imidazole or a phosphine. As a more preferred polymerization catalyst, tetrabutylphosphoniumbromide may be listed.

The amount of polymerization catalyst added varies depending on the components, the mixing ratio and the polymerization and curing method of the composition, and cannot be generalized. Generally, it is preferably 0.03% to 0.5% by mass relative to the total amount of the optical material composition. When the amount of polymerization catalyst added is larger than 1% by mass, rapid polymerization may occur. When the amount of polymerization catalyst added is less than 0.01%, the optical material composition may not be fully cured and the heat resistance may be poor.

In addition, when producing the corresponding optical material, adding additives to the above-mentioned polymerizable and curable composition can further improve the usability of the obtained optical material. That is, the polymerizable and curable composition of the present invention can also contain additives such as an ultraviolet absorber, a mold release agent, a bluing agent and a red agent; wherein the ultraviolet absorber is selected from the benzotriazole compounds, in particular preferably 2-(2-hydroxy-tert-octylphenyl)-2H-benzotriazole and 2-(2-hydroxy-5-t-octylphenyl)-2H-benzotriazole, and the addition amount is 0.001%-1% by mass of the total amount for the polymerizable and curable composition, and more preferably 0.01%-0.5% by mass; the bluing agent and the red agent are added according to the actual needs of the optical material, and there is no specific requirement for the addition amount; and the mold release agent is selected from one or more of di-n-butyl phosphate, El310, polyoxyethylene nonyl phenyl ether phosphate, and Zelec UNTM, and the addition amount is 0.001% to 1% by mass of the total amount for the polymerizable and curable composition, more preferably 0.01% to 0.5% by mass.

The present invention also provides an optical material, which is obtained by curing the above-mentioned polymerizable and curable composition, and the specific steps are as follows:
a) Mix the compositions for optical material uniformly to obtain a reaction mixture;
b) Inject the reaction mixture obtained in step a) into a mold through filter membrane for the first curing. After the mold release, carry out edge grinding and multi-slot ultrasonic cleaning. After the cleaning is completed, conduct the second curing to obtain an optical resin material.

Wherein, a step of raising the temperature during the first curing in step b) is as follows: the initial temperature is 15° C. to 25° C., keep the initial temperature for 2.0 hours to 3.5 hours, then raise the temperature to 45° C. to 60° C. over 10 hours to 15 hours, then raise the temperature to 75° C. to 90° C. over 2.0 hours to 4.0 hours, and finally lower the temperature to 60° C. to 75° C. over 1.5 hours to 2.5 hours; and the temperature during the second curing in step b) is 80° C. to 110° C., and the curing time is 2 hours to 4 hours.

The process parameters for multi-slot ultrasonic cleaning are as follows:

| Cleaning Slot | | | Temperature |
|---|---|---|---|
| Position | Additive | Addition Amount | (° C.) |
| Feed-in slot | Alkaline solution | 3% or 6% | 48 ± 2 |
| 1-4 | Cleaning agent | 100% | 48 ± 2 |
| 5 | / | | |
| 6 | Ultrapure water | 100% | 48 ± 2 |
| 7 | Ultrapure water + | 100% + 80 mL | 48 ± 2 |
| | cleaning agent | | |
| 8-9 | Ultrapure water | 100% | 48 ± 2 |
| 10 | Ultrapure water | 100% | 55 ± 3 |
| 11 | Sprinkling | | |
| 12-15 | Deionized water | 100% | 55 ± 3 |
| 16 | Deionized water | 100% | 55 ± 3 |
| 17 | Deionized water | 100% | 75 ± 5 |
| Drying tunnel | / | / | 90 ± 2 |

| | |
|---|---|
| Cleaning time | 135s/station |

For the specific cleaning process and the reagents used, refer to the methods and reagents documented in the existing Chinese patent of CN104802430B.

The optical material obtained above can be used in the preparation of optical lenses.

To sum up, compared with the prior art, the sulfoxide structure is introduced into the episulfide compound obtained in the present application, and the presence of the polar group can effectively improve the alkali corrosion resistance of optical material substrates and reduce the rate of substrate burn, thereby improving substrate yield and reducing production costs.

### BRIEF DESCRIPTION OF THE DRAWINGS

FIG. 1 is a mass spectrum of the episulfide compound in Embodiment 1;

FIG. 2 is a ¹H NMR spectrum of the episulfide compound in Embodiment 1;

FIG. 3 is a ¹³C NMR spectrum of the episulfide compound in Embodiment 1.

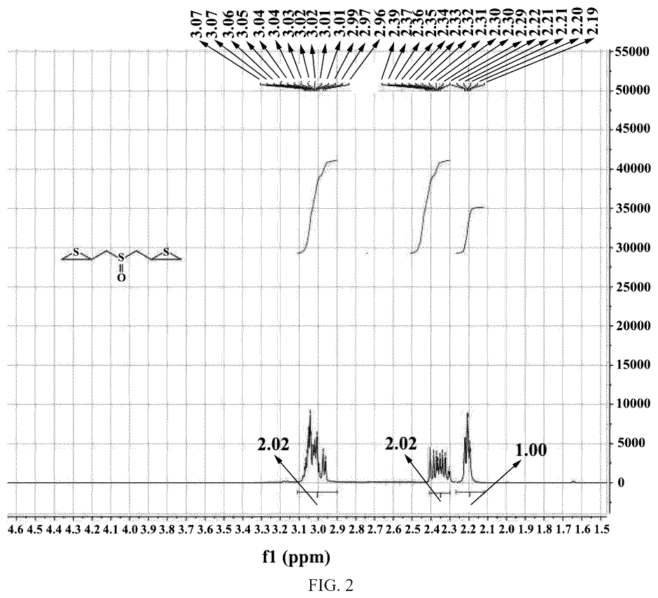

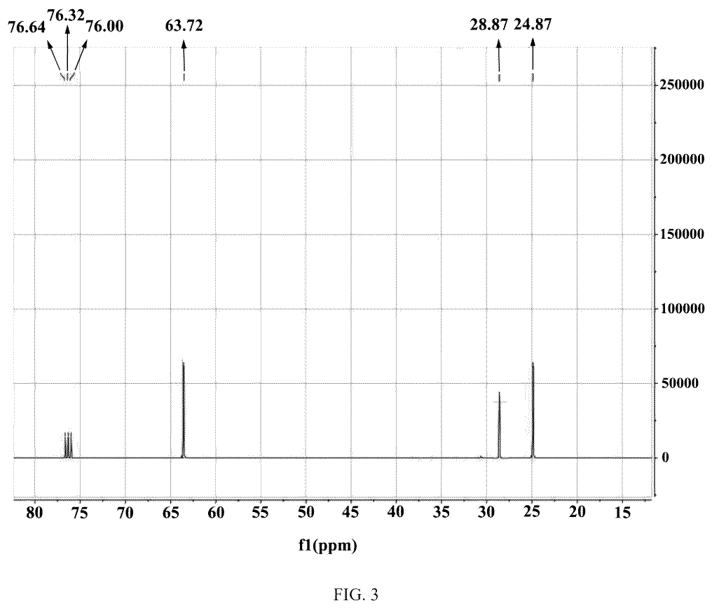

In FIG. 2 and FIG. 3, ¹HNMR (CDCl₃) δ=2.21 ppm (1H), δ=2.32 ppm (2H), δ=3.02 ppm (2H); ¹³CNMR (CDCl₃) δ=24.8 ppm, 28.6 ppm, 63.7 ppm.

### DETAILED DESCRIPTION OF THE EMBODIMENTS

The above content of the present invention is further detailed through the specific embodiments, but this should not be understood as the scope of the above subject matter of the present invention being limited to the following embodiments. All technologies realized on the basis of the above content for the present invention belong to the scope of the present invention, and unless otherwise specified, the raw materials used in the following embodiments are all commercially available products.

In order to further illustrate the present invention, the following embodiments are provided for detailed description.
1) Rate of substrate burn: “substrate burn” refers to the phenomenon that impurity spots are generated around the midpoint of the substrate due to the influence of alkali concentration or temperature during the substrate cleaning process. In the embodiments, 100 substrates subjected to ultrasonic cleaning are visually observed to determine the burned substrates, thereby calculating the rate of substrate burn.
2) Yield rate: Product A has no impurity spots within the center radius of 3 cm; Product B has no impurity spots within the center radius of 1.5 cm, and has impurity spots within 1.5-3 cm; and Product C has impurity spots within the center radius of 1.5 cm. Among them, products A and B are qualified products, and product C is a non-conforming product. In the embodiments, 100 substrates subjected to ultrasonic cleaning were visually observed to determine whether there are impurity spots within different center radii, thereby calculating the yield rate.

### Embodiment 1

A method for preparing the episulfide compound represented by formula (1):

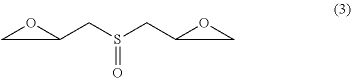

81 g (0.5 mol) of the compound represented by formula (3), 500 mL of methanol, 500 mL of toluene, 87.4 g (1.15 mol) of thiourea and 6 g (0.1 mol) of acetic acid were put for reaction at 30° C. for 12 hours. Then, a toluene was put for extraction. The obtained organic layer was washed with with water, and the solvent was removed by distillation. The crude product was conventionally separated and refined by silica gel column to obtain 48.5 g (0.25 mol) of the episulfide compound represented by formula (1).

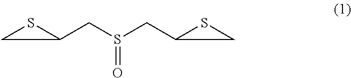

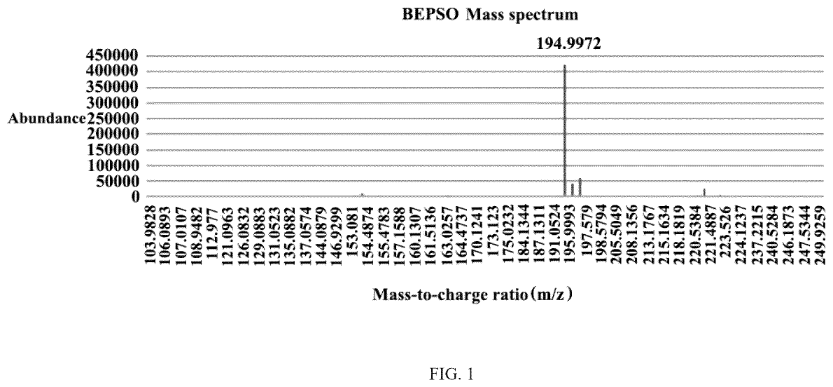

The episulfide compound represented by formula (1) was characterized by mass spectrometry and nuclear magnetic resonance, and the results are shown in FIGS. 1-3:

Mass spectrum (ESI): [M+H]⁺=194.9972.

### Embodiment 2

88.4 g of bis(β-epithiopropyl)sulfide, 0.1 g of the episulfide compound represented by formula (1), 5.1 g of isophoronediisocyanate, 6.3 g of mercaptoethanol, 0.1 g of tetrabutylphosphoniumbromide, 0.3 g of 2-(2-hydroxy-tert-octylphenyl)-2H-benzotriazole (UV-329), and 0.2 g of di-n-butyl phosphate were mixed and stirred for 50 minutes to obtain a prepolymer liquid. The density ρ of the prepolymer liquid before curing was measured by a liquid density meter, the obtained prepolymer liquid was degassed in vacuum for 30 min, filtered by polytetrafluoroethylene filter membrane with a pore size of 3 μm and injected into a glass mold, and then the mold was put into a temperature-programmed curing furnace for the first curing; as a result, a resin lens after the first curing was obtained. The temperature procedure for the first curing is that: the initial temperature is 20° C., keep at 20° C. for 2 hours, then raise the temperature to 45° C. over 3.5 hours, then raise the temperature to 55° C. over 3 hours, then raise the temperature to 100° C. over 6 hours, keep at 100° C. for 4 hours, and finally lower the temperature to 70°° C. over 2 hours.

The obtained resin lens substrate after the first curing was released from the mold and was subjected to multi-slot ultrasonic cleaning, after cleaning, the substrate was visually observed, and the rate of substrate burn and the yield rate were calculated and determined.

The process parameters for multi-slot ultrasonic cleaning are as follows:

| Cleaning Slot | | | Temperature |
|---|---|---|---|
| Position | Additive | Addition Amount | (° C.) |
| Feed-in slot | Alkaline solution | 3% or 6% | 48 ± 2 |
| 1-4 | Cleaning agent | 100% | 48 ± 2 |
| 5 | / | | |
| 6 | Ultrapure water | 100% | 48 ± 2 |
| 7 | Ultrapure water + | 100% + 80 mL | 48 ± 2 |
| | cleaning agent | | |
| 8-9 | Ultrapure water | 100% | 48 ± 2 |
| 10 | Ultrapure water | 100% | 55 ± 3 |
| 11 | Sprinkling | | |
| 12-15 | Deionized water | 100% | 55 ± 3 |
| 16 | Deionized water | 100% | 55 ± 3 |
| 17 | Deionized water | 100% | 75 ± 5 |
| Drying tunnel | / | / | 90 ± 2 |
| Cleaning time | 135s/station | | |

For the specific cleaning process and the reagents used, refer to the methods and reagents documented in the existing Chinese patent of CN104802430B. Among them, the concentration of the alkaline solution is 3% or 6%.

### Embodiment 3

The difference from Embodiment 2 is that: 87.5 g of bis(β-epithiopropyl)sulfide and 1.0 g of the episulfide compound represented by formula (1) were added, and the addition amounts or processes of other substances were the same as that in Embodiment 2.

### Embodiment 4

The difference from Embodiment 2 is that: 86.5 g of bis(β-epithiopropyl)sulfide and 2.0 g of the episulfide compound represented by formula (1) were added, and the addition amounts or processes of other substances were the same as that in Embodiment 2.

### Embodiment 5

The difference from Embodiment 2 is that: 85.5 g of bis(β-epithiopropyl)sulfide and 3.0 g of the episulfide compound represented by formula (1) were added, and the addition amounts or processes of other substances were the same as that in Embodiment 2.

### Embodiment 6

The difference from Embodiment 2 is that: 88.4 g of bis(β-epithiopropyl)sulfide and 0.1 g of the episulfide compound represented by formula (1) were added, the concentration of the alkaline solution for multi-slot ultrasonic cleaning was 6% after the first curing, release and demolding, and the addition amounts or processes of other substances were the same as that in Embodiment 2.

### Embodiment 7

The difference from Embodiment 2 is that: 87.5 g of bis(β-epithiopropyl)sulfide and 1.0 g of the episulfide compound represented by formula (1) were added, the concentration of the alkaline solution for multi-slot ultrasonic cleaning was 6% after the first curing, release and demolding, and the addition amounts or processes of other substances were the same as that in Embodiment 2.

### Embodiment 8

The difference from Embodiment 2 is that: 86.5 g of bis(β-epithiopropyl)sulfidee and 2.0 g of the episulfide compound represented by formula (1) were added, the concentration of the alkaline solution for multi-slot ultrasonic cleaning was 6% after the first curing, release and demolding, and the addition amounts or processes of other substances were the same as that in Embodiment 2.

### Embodiment 9

The difference from Embodiment 2 is that: 85.5 g of bis(β-epithiopropyl)sulfide and 3.0 g of the episulfide compound represented by formula (1) were added, the concentration of the alkaline solution for multi-slot ultrasonic cleaning was 6% after the first curing, release and demolding, and the addition amounts or processes of other substances were the same as that in Embodiment 2.

### Comparative Embodiment 1

The difference from Embodiment 2 is that: 88.5 g of bis(β-epithiopropyl)sulfide and 0 g of the episulfide compound represented by formula (1) were added, and the addition amounts or processes of other substances were the same as that in Embodiment 2.

### Comparative Embodiment 2

The difference from Embodiment 2 is that: 84.5 g of bis(β-epithiopropyl)sulfide and 4 g of the episulfide compound represented by formula (1) were added, and the addition amounts or processes of other substances were the same as that in Embodiment 2.

### Comparative Embodiment 3

The difference from Embodiment 2 is that: 82.5 g of bis(β-epithiopropyl)sulfide and 6 g of the episulfide compound represented by formula (1) were added, and the addition amounts or processes of other substances were the same as that in Embodiment 2.

### Comparative Embodiment 4

The difference from Embodiment 2 is that: 88.5 g of bis(β-epithiopropyl)sulfide and 0 g of the episulfide compound represented by formula (1) were added, the concentration of the alkaline solution for multi-slot ultrasonic cleaning was 6% after the first curing, release and demolding, and the addition amounts or processes of other substances were the same as that in Embodiment 2.

### Comparative Embodiment 5

The difference from Embodiment 2 is that: 84.5 g of bis(β-epithiopropyl)sulfide and 4 g of the episulfide compound represented by formula (1) were added, the concentration of the alkaline solution for multi-slot ultrasonic cleaning was 6% after the first curing, release and demolding, and the addition amounts or processes of other substances were the same as that in Embodiment 2.

### Comparative Embodiment 6

The difference from Embodiment 2 is that: 82.5 g of bis(β-epithiopropyl)sulfidee and 6 g of the episulfide compound represented by formula (1) were added, the concentration of the alkaline solution for multi-slot ultrasonic cleaning was 6% after the first curing, release and demolding, and the addition amounts or processes of other substances were the same as that in Embodiment 2.

Embodiments and comparative embodiments were used to calculate the rate of substrate burn and the yield rate, and the specific results are shown in the table below.

| | | Concentration | | | | |
|---|---|---|---|---|---|---|
| | | of the | | Addition | Rate of | |
| | | alkaline | Component | Amount (% | Substrate | Yield |
| | Main Component | solution | Added | by mass) | Burn | Rate |
| Embodiment | bis(β-epithiopropyl)sulfide | 3% | BEPSO | 0.1 | 12% | 95% |
| 2 | | | | | | |
| Embodiment | bis(β-epithiopropyl)sulfide | | BEPSO | 1.0 | 10% | 96% |
| 3 | | | | | | |
| Embodiment | bis(β-epithiopropyl)sulfide | | BEPSO | 2.0 | 10% | 95% |
| 4 | | | | | | |
| Embodiment | bis(β-epithiopropyl)sulfide | | BEPSO | 3.0 | 8% | 97% |
| 5 | | | | | | |
| Embodiment | bis(β-epithiopropyl)sulfide | 6% | BEPSO | 0.1 | 12% | 94% |
| 6 | | | | | | |
| Embodiment | bis(β-epithiopropyl)sulfide | | BEPSO | 1.0 | 10% | 95% |
| 7 | | | | | | |
| Embodiment | bis(β-epithiopropyl)sulfide | | BEPSO | 2.0 | 9% | 96% |
| 8 | | | | | | |
| Embodiment | bis(β-epithiopropyl)sulfide | | BEPSO | 3.0 | 8% | 98% |
| 9 | | | | | | |
| Comparative | bis(β-epithiopropyl)sulfide | 3% | BEPSO | 0 | 20% | 85% |
| embodiment | | | | | | |
| 1 | | | | | | |
| Comparative | bis(β-epithiopropyl)sulfide | | BEPSO | 4.0 | 9% | 96% |
| embodiment | | | | | | |
| 2 | | | | | | |
| Comparative | bis(β-epithiopropyl)sulfide | | BEPSO | 6.0 | 8% | 97% |
| embodiment | | | | | | |
| 3 | | | | | | |
| Comparative | bis(β-epithiopropyl)sulfide | 6% | BEPSO | 0 | 25% | 83% |
| embodiment | | | | | | |
| 4 | | | | | | |
| Comparative | bis(β-epithiopropyl)sulfide | | BEPSO | 4.0 | 10% | 96% |
| embodiment | | | | | | |
| 5 | | | | | | |
| Comparative | bis(β-epithiopropyl)sulfide | | BEPSO | 6.0 | 9% | 97% |
| embodiment | | | | | | |
| 6 | | | | | | |

In the above table, BEPSO is the episulfide compound represented by formula (1).

It can be seen from the results in the above table that, when the addition amount of the episulfide compound represented by formula (1) is 0.1 to 3.0% by mass, the rate of substrate burn for the optical resin material lens is about 10%, the substrate yield rate is greater than 95%, and the stability is optimal; when the addition amount of the episulfide compound represented by formula (1) is greater than 3%, the rate of substrate burn and the yield rate do not change significantly; and when the addition amount is 0, the rate of substrate burn is greater than 20%, and the yield rate is less than 90%, which increases the cost.

The above embodiments enable those skilled in the field to implement or use the present invention. Various modifications to these embodiments will be apparent to those skilled in the field, and the general principles defined herein may be implemented in other embodiments without departing from the spirit or scope of the present invention. Therefore, the present invention will not be limited to the embodiments shown herein, but shall conform to the widest scope consistent with the principles and novel features disclosed herein.

What is claimed is:

1. A novel episulfide compound, wherein the novel episulfide compound is represented by formula (1):

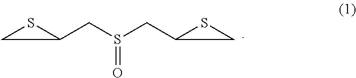

2. An optical material composition, comprising a polymerizable compound, wherein the polymerizable compound mainly composed of the episulfide compound represented by formula (1) and a compound represented by formula (2): 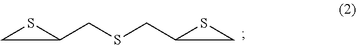
wherein the episulfide_compound represented by formula (1) accounts for 0.001% to 6.0% by mass of a total amount of the optical material composition.

3. The optical material composition according to claim 2, wherein the optical material composition further comprises a thiol compound and an isocyanate compound;
the episulfide compound represented by formula (1) accounts for 0.1% to 3.0% by mass of the total amount of the optical material composition.

4. The optical material composition according to claim 2, wherein the compound represented by formula (2) accounts for 50.0% to 99.999% by mass of the total amount of the optical material composition.

5. The optical material composition according to claim 4, wherein the compound represented by formula (2) accounts for 70.0% to 99.999% by mass of the total amount of the optical material composition.

6. A polymerizable and curable composition, comprising the optical material composition according to claim 2 and a polymerization catalyst at a content of 0.01% to 1% by mass with respect to the total amount of the optical material composition.

7. The polymerizable and curable composition according to claim 6, wherein the polymerization catalyst is an imidazole or a phosphine, the polymerization catalyst is contained at a content of 0.03% to 0.5% by mass with respect to the total amount of the optical material composition.

8. An optical material, wherein the optical material is obtained by curing the polymerizable and curable composition according to claim 6.

9. An optical lens, wherein the optical lens is prepared from the optical material according to claim 8.

10. The optical material composition according to claim 3, wherein the compound represented by formula (2) accounts for 50.0% to 99.999% by mass of the total amount of the optical material composition.

11. The polymerizable and curable composition according to claim 6, wherein the optical material composition further comprises a thiol compound and an isocyanate compound; the episulfide compound represented by formula (1) accounts for 0.1% to 3.0% by mass of the total amount of the optical material composition.

12. The polymerizable and curable composition according to claim 6, wherein in the optical material composition, the compound represented by formula (2) accounts for 50.0% to 99.999% by mass of the total amount of the optical material composition.

13. The polymerizable and curable composition according to claim 12, wherein in the optical material composition, the compound represented by formula (2) accounts for 70.0% to 99.999% by mass of the total amount of the optical material composition.

14. The optical material according to claim 8, wherein in the polymerizable and curable composition, the polymerization catalyst is an imidazole or a phosphine, the polymerization catalyst is contained at a content of 0.03% to 0.5% by mass with respect to the total amount of the optical material composition.

15. The optical material composition according to claim 10, wherein the compound represented by formula (2) accounts for 70.0% to 99.999% by mass of the total amount of the optical material composition.

16. The polymerizable and curable composition according to claim 11, wherein the polymerization catalyst is an imidazole or a phosphine, the polymerization catalyst is contained at a content of 0.03% to 0.5% by mass with respect to the total amount of the optical material composition.

17. The polymerizable and curable composition according to claim 12, wherein the polymerization catalyst is an imidazole or a phosphine, the polymerization catalyst is contained at a content of 0.03% to 0.5% by mass with respect to the total amount of the optical material composition.

18. The polymerizable and curable composition according to claim 13, wherein the polymerization catalyst is an imidazole or a phosphine, the polymerization catalyst is contained at a content of 0.03% to 0.5% by mass with respect to the total amount of the optical material composition.

19. An optical lens, wherein the optical lens is prepared from the optical material according to claim 14.
